(19) United States
(12) Patent Application Publication
(10) Pub. No.: US 2015/0289750 A1
(43) Pub. Date: Oct. 15, 2015

(54) IMAGING AND TREATMENT DEVICE

(71) Applicants: Jeremy Stigall, San Diego, CA (US); Emmett Kearney, San Diego, CA (US)

(72) Inventors: Jeremy Stigall, San Diego, CA (US); Emmett Kearney, San Diego, CA (US)

(21) Appl. No.: 14/683,747

(22) Filed: Apr. 10, 2015

Related U.S. Application Data
(60) Provisional application No. 61/978,320, filed on Apr. 11, 2014.

(51) Int. Cl. A61B 1/00; A61B 18/02; A61M 25/09; A61B 1/005; A61B 8/12; A61B 5/00; A61B 18/14; A61N 7/02; A61B 1/313; A61B 8/00

(52) U.S. Cl. CPC A61B 1/00087; A61B 1/3137; A61B 18/02; A61M 25/09; A61B 8/445; A61B 8/12; A61B 5/0066; A61B 5/0084; A61B 8/4483; A61B 1/00082; A61B 1/00045; A61B 18/1492; A61N 7/022; A61B 1/0057; A61B 2018/0212; A61M 2025/0177

(57) ABSTRACT

A medical device includes both intraluminal imaging and denervation capabilities to allow guided delivery of denervation therapy to target tissues with a single device and in order to treat diseases such as hypertension.

### RELATED APPLICATIONS

This application claims the benefit of and priority to U.S. Provisional Patent Application Ser. No. 61/978,320, filed Apr. 11, 2014, the entirety of which is incorporated by reference herein.

### FIELD OF THE INVENTION

The invention relates to medical devices and systems for use in, for example, renal denervation.

### BACKGROUND

Hypertension is one of the most prevalent cardiovascular risk factors, afflicting 34% of adults worldwide and is a leading cause of mortality worldwide. Due to noncompliance to pharmacological therapy or resistance to medical therapy, only a small sub-group of afflicted adults have hypertension under control. The renal sympathetic nervous system has been identified as a major contributor to the complex pathophysiology of hypertension, states of volume overload (such as heart failure) and progressive renal disease. Disruption of these renal sympathetic nerves has positive effects on hypertension and other diseases, such as sleep apnea, insulin resistance, and metabolic changes in polycystic ovary syndrome. The renal sympathetic efferent and afferent nerves are positioned within and immediately adjacent to the wall of the renal artery, and have a crucial role in sympathetic nervous system signaling and activation. Thus, the interior lumen of the renal artery is a targeted located for treatment applications and procedures.

Renal sympathetic denervation (RDN) is a method of treatment for diseases such as hypertension and is performed by delivering high frequency energy within the lumen of the renal arteries to disrupt the network of renal afferent and efferent nerves. Commonly, renal denervation procedures involve the delivery of radio frequency (RF) to the interior lumen of the renal artery. For example, once a catheter is positioned in the renal artery, the tissue is treated by applying RF, and each RF application is followed by retraction by at least 5 mm and rotation by 90 degrees of the catheter tip from the first distal main renal artery bifurcation to the ostium. The process is repeated until the nerves are effectively treated.

Visualization of tissues during renal sympathetic denervation procedures requires the application of externally applied imaging modalities, such as fluoroscopy or by venography and angiography. Venography and angiography require the injection of contrast dyes into the patient for visualization of the anatomy of the renal arteries using an externally applied x-ray imaging modality. During this procedure, the patient and the medical staff are exposed to radiation, which can increase the chances of cancer and other radiation concerns. In addition, guiding the catheter and relying on these visualization means can lead to error, including insufficient treatment application or over-treatment.

### SUMMARY

The invention generally relates to medical devices, systems, and methods for providing denervation therapy utilizing a single catheter with both denervation and intraluminal imaging capabilities. When used for renal denervation, the intraluminal imaging capability can provide an accurate, real-time depiction of the target tissue to allow for precise positioning of the denervation assembly relative to the renal afferent and efferent nerves and to assess the progress of the renal denervation procedure. Aside from renal denervation therapy, the devices and systems of the invention are broadly applicable to any ablative procedure, i.e., wherein the energy level within a tissue is altered to affect a therapeutic change.

The invention recognizes that current intraluminal imaging and interventional techniques do not allow for real-time imaging of the internal lumen of the vessel during a treatment procedure. By contrast, devices and systems of the invention utilize an onboard imaging module capable of locating clusters of afferent and efferent nerves during the denervation procedure; providing a more accurate image of the target tissue while eschewing the need for prolonged exposure to the radiation and contrast media found in the imaging techniques currently employed in denervation.

Furthermore, aspects of the present invention reduce the risk of ineffective delivery of treatment due to inaccurate detection and visualization of afferent and efferent nerves in the renal artery. The onboard imaging capabilities allow for real-time imaging of the intralumenal spaces of arteries and the denervation assembly allows for focused delivery of denervation to a selected region of interest once visually located. Real-time visualization of the arterial walls allows for precise placement of the denervation assembly, minimizing possible damage to the kidneys and surrounding vessels. After application, the onboard imaging capabilities allow the treated tissue to be analyzed in order to determine if further treatment is needed, thereby preventing excessive application and the risks associated therewith.

The devices and systems of the invention can be used in conjunction with an imaging assembly such as optical coherence tomography (OCT) or intravascular ultrasound (IVUS). Denervation techniques utilized by the devices and systems of the invention may include application of radiofrequency (RF) energy or high intensity focused ultrasound energy to heat the target tissue or delivery of a refrigerant to cool the target tissue to the point of denervation. Using the disclosed devices and systems of the invention allows for safer denervation procedures for the patient and the medical staff as well as shorter procedure times, more accurate application of denervation therapy, and real-time verification of results.

### BRIEF DESCRIPTION OF THE DRAWINGS

FIG. 1 shows a catheter assembly including a catheter body, and an actuator for manually or automatically controlling an element of the catheter.

FIG. 2 shows a catheter assembly comprising a multi-lumen catheter, an imaging assembly, a denervation assembly, and a controller to control the imaging and denervation assemblies of the catheter.

FIG. 3 shows the distal end of a catheter within a lumen and including a denervation assembly and an imaging assembly on a drive cable within the catheter.

FIG. 4 shows a cross section view of a catheter of the invention with a drive cable, a guidewire lumen, and a supply and return lumen for the circulation of refrigerant to a denervation assembly on the distal portion of the catheter.

FIG. 5 shows an embodiment of a catheter of the invention with an imaging assembly on an internal drive cable and an expandable basket member with a denervation assembly thereon.

FIG. 6 shows an embodiment of a catheter of the invention within a lumen having an imaging assembly on the catheter body and an expanded balloon member with a denervation assembly thereon.

### DETAILED DESCRIPTION

The invention generally relates to imaging and treatment devices and systems to provide denervation therapy to a patient (e.g., renal denervation). In certain aspects, the invention provides a catheter with both an imaging and denervation assembly. The denervation assembly may be positioned on a distal end of the catheter body and is configured to deliver or remove energy from the target tissue to effect denervation. In certain aspects, an imaging assembly is positioned on a drive cable within a lumen in the catheter body and may be moved relative to the catheter body to provide intralumenal imaging for guidance and feedback during denervation treatment.

### Catheter

In certain embodiments, the device is a catheter and configured for intraluminal introduction to a target body lumen, such as the renal artery. The dimensions and other physical characteristics of the catheter bodies will vary significantly depending on the body lumen that is to be accessed. In particular, catheters can be intended for “over-the-wire” introduction when a guidewire channel extends fully through the catheter body or for “rapid exchange” introduction where the guidewire channel extends only through a distal portion of the catheter body. In other cases, it may be possible to provide a fixed or integral coil tip or guidewire tip on the distal portion of the catheter or even dispense with the guidewire entirely. For convenience of illustration, guidewires will not be shown in all embodiments, but it should be appreciated that they can be incorporated into any of these embodiments.

Catheter bodies intended for intravascular introduction will typically have a length in the range from 50 cm to 200 cm and an outer diameter in the range from 1 French to 12 French (0.33 mm: 1 French), usually from 3 French to 9 French. Catheter bodies will typically be composed of an organic polymer that is fabricated by conventional extrusion techniques. Suitable polymers include polyvinylchloride, polyurethanes, polyesters, polytetrafluoroethylenes (PTFE), silicone rubbers, natural rubbers, and the like. Optionally, the catheter body may be reinforced with braid, helical wires, coils, axial filaments, or the like, in order to increase qualities such as rotational strength, column strength, toughness, or pushability. Suitable catheter bodies may be formed by extrusion, with one or more channels being provided when desired. The catheter diameter can be modified by heat expansion and shrinkage using conventional techniques. The resulting catheters will thus be suitable for introduction to the vascular system, often the coronary arteries, by conventional techniques.

In some embodiments of the invention, the distal portion of the catheter body of the present invention may have a wide variety of forms and structures. In many embodiments, a distal portion of the catheter comprises transducers for imaging. In some embodiments, the distal portion may be more rigid than a proximal portion, but in other embodiments the distal portion may be equally as flexible as the proximal portion. One aspect of the present invention provides catheters having a lumen. In some embodiments, the lumen of the catheter contains a drive cable that comprises an imaging assembly. In other embodiments, a lumen of the catheter contains a refrigerant channel to supply a denervation assembly on the distal end of the catheter body and in fluid communication with the refrigerant channel. In most embodiments a rigid distal portion or housing of the catheter body will have a diameter that generally matches the proximal portion of the catheter body, however, in other embodiments, the distal portion may be larger or smaller than the proximal portion of the catheter. A rigid distal portion of a catheter body can be formed from materials that are rigid or which have very low flexibilities, such as metals, hard plastics, composite materials, NiTi, steel with a coating such as titanium nitride, tantalum, ME-92 (antibacterial coating material), or diamonds. Most usually, the distal end of the catheter body will be formed from stainless steel or platinum/iridium.

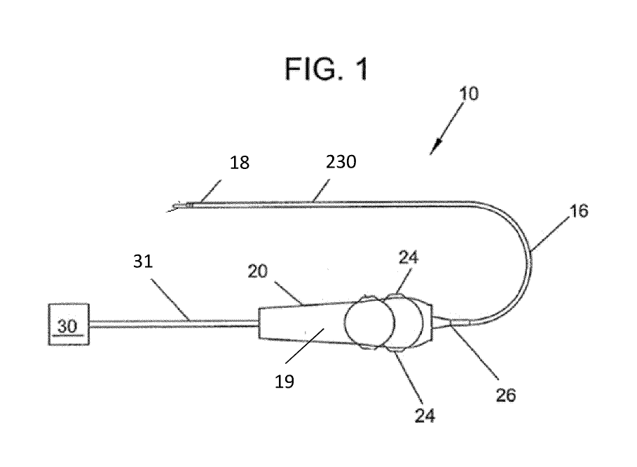

FIG. 1 illustratively depicts an embodiment of the catheter system 10 including a catheter body 230. The catheter body 230 is a generally elongate member having a distal segment 18, a proximal segment 16, and at least one lumen (not shown). In some embodiments, a drive cable (not shown) is disposed within the lumen of the catheter body. The proximal segment 16 is attached to a handle 19. The handle 19 includes, by way of example, a housing 20, and an actuator 24.

The actuator 24 is manipulated by a user moving an exposed control surface of the actuator 24 (using a finger/thumb) lengthwise along the length of the housing 20 of the handle 19 (as opposed to across the width of the handle 19). In alternative embodiments, thumb-controlled slider actuators replace the rotating knobs. The catheter body 230 is made, by way of example, of engineered nylon (polyether block amide) and includes a tube or tubing, alternatively called a catheter tube or catheter tubing that has at least one lumen.

In the illustrative example in FIG. 1, the actuator 24 is accessible (have exposed control surfaces through the housing 20) on two sides of the handle 19. A strain relief 26 protects the catheter body 230 at a point where the catheter body proximal segment 16 meets the handle 19. A cable 31 connects the handle 19 to a connector 30. The connector 30, which can be any of many possible configurations, is configured to interconnect with an imaging system for processing, storing, manipulating, and displaying data obtained from signals generated by a sensor mounted at the distal segment 18 of the catheter body 230.

In one embodiment, the actuator 24 controls the drive cable positioned within the lumen of the catheter body. The user's manipulation of the actuator 24 controls the position of the drive cable by sliding within the lumen, and by rotating the drive cable about its central axis.

In another embodiment, the actuator 24, or another actuator disposed on the housing 20, controls the catheter body. The user's manipulation of the actuator 24 controls the position of the distal end of the catheter. In certain embodiments, the drive cable may be manipulated by a controller.

It should be appreciated that the device of the invention can be used in conjunction with an imaging guidewire, which can be introduced into a lumen of the body to obtain real-time images of the vessel prior to introduction of a catheter. The body lumens generally are diseased body lumens and, in particular, lumens of the vasculature. The real-time images obtained may be used to locate a region or location of interest within a body lumen. Regions of interest are typical regions that include a defect or tissues requiring treatment. The devices and methods, however, are also suitable for treating stenosis of body lumens and other hyperplastic and neoplastic conditions in other body lumens, such as the ureter, the biliary duct, respiratory passages, the pancreatic duct, the lymphatic duct, and the like. In addition, the region of interest can include, for example, a location for stent placement or a location including plaque or diseased tissue that needs to be removed or treated. In some instances, the region of interest may include the renal artery where renal denervation therapy may be applied to the afferent and efferent nerves therein.

In embodiments incorporating an imaging guidewire, once the imaging guidewire is in place, the catheter can be introduced over the guidewire to the location of interest. The imaging assembly can obtain images of the intraluminal surface as the imaging assembly and catheter moves towards the region of interest, which allows the denervation assembly of the catheter to be precisely placed into the region of interest and provides for tracking of the catheter along the path of the guidewire. In addition, the imaging assembly of the catheter can be used to obtain different imaging views of the region of interest. For example, the imaging assembly can be used to locate the renal artery and afferent and efferent nerve clusters found therein.

In certain aspects, the catheter may also serve as a delivery catheter, ablation catheter, extraction catheter or energizing catheter to perform an intraluminal procedure. The catheter may include a denervation assembly to perform an intraluminal procedure. During the procedure, both the imaging guidewire and the imaging assembly may be used to image cross-sections of the luminal surface. In addition, the catheter may also include forward or distal facing imaging assemblies to image the luminal space and/or any procedure in front of or distal to the catheter. For example, the imaging assembly can axially image a luminal surface for the location and selection of a region of interest suspected of containing afferent and efferent nerves for the accurate and targeted delivery of a treatment. This greatly improves visualization during the procedure by allowing an operator to have real-time images of the vessel wall while the denervation assembly of the catheter is engaged with that portion of the vessel wall. After the treatment procedure, the imaging assembly of the catheter can be used to perform a final visualization of the luminal surface before the catheter is removed from the patient.

The devices of the invention may include static imaging assemblies that do not move with respect to the catheter body, or moving imaging assemblies. For example, the imaging assembly may be a phased array of ultrasonic transducers for IVUS imaging, or a collection of CCD arrays. An array of assemblies will typically cover a circumference of the catheter to provide a 360° view of the lumen.

In other embodiments, the imaging assembly may rotate or translate using drive cables within the catheter body. Catheters having imaging assemblies that rotate and translate are known generally as “pull-back” catheters. The principles of pull-back OCT are described in detail in U.S. Pat. No. 7,813,609 and US Patent Publication No. 20090043191, both of which are incorporated herein by reference in their entireties. The mechanical components, including drive shafts, rotating interfaces, windows, and couplings, are similar between the various forms of pull-back imaging.

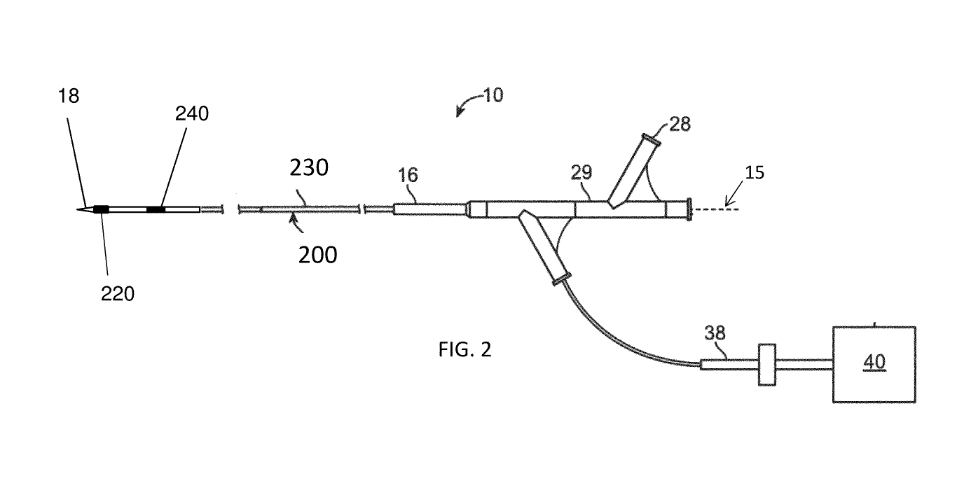

Another embodiment of a catheter system 10 for use with the invention is shown in FIG. 2. FIG. 2 is merely exemplary, as many other configurations of the catheter system 10 are possible to achieve the principles of the invention, i.e., imaging and treating a lumen. The catheter system 10 includes a catheter 200 having a catheter body 230 with a proximal end 16 and a distal end 18. The catheter body 230 is flexible and defines a catheter axis 15, and may include one or more lumens, such as a guidewire lumen, a drive cable lumen, or a refrigerant supply lumen. The catheter 200 also includes an imaging assembly 240, and a denervation assembly 220, on the distal end 18. On the proximal end 16, the catheter 200 has a housing 29. As discussed previously and described below, the imaging assembly 240 may comprise any of a number of imaging devices. The denervation assembly 220 may comprise any number of denervation devices as described below. In some embodiments, housing 29 includes a connector 28 in fluid communication with the lumen of the catheter body 230. Connectors, such as 26 and 28, may optionally comprise standard connectors, such as Luer-Loc™ connectors.

Housing 29 also accommodates electrical or optoelectrical connectors 38 for powering the imaging assembly and receiving the reflected/scattered light. Connector 38 includes a plurality of electrical connections, each electrically coupled the imaging assembly 240. In some embodiments, the connector 38 is also a mechanical connector in addition to an electrical or optoelectric connector. The mechanical connector can be used to rotate and translate the imaging assembly 240.

The controller 40 includes a processor, or is coupled to a processor, to control and/or record treatment. The controller will typically comprise computer hardware and/or software, often including one or more programmable processor units running machine readable program instructions or code for implementing some or all of one or more of the methods described herein. The code will often be embodied in a tangible media such as a memory (optionally a read only memory, a random access memory, a non-volatile memory, or the like) and/or a recording media (such as a floppy disk, a hard drive, a CD, a DVD, a non-volatile solid-state memory card, or the like). The code and/or associated data and signals may also be transmitted to or from the processor via a network connection, and some or all of the code may also be transmitted between components of catheter system 10 and within the controller 40.

In certain embodiments, the controller may direct rotational or longitudinal movement of an imaging assembly on the catheter body or on a drive cable. The controller can be configured to receive and display imaging data from the imaging assembly and to coordinate intraluminal movements of the imaging assembly while receiving data (e.g., in pull-back IVUS or pull-back OCT). Furthermore, the controller may also control movement and activation of the denervation assembly to facilitate placement of the denervation assembly in relation to the target tissue and delivery of denervation therapy to the target tissue. In certain embodiments, the controller may control deployment of an expandable member in order to bring a denervation assembly mounted thereon into contact with target tissue on the wall of the lumen (e.g., renal denervation in a renal artery).

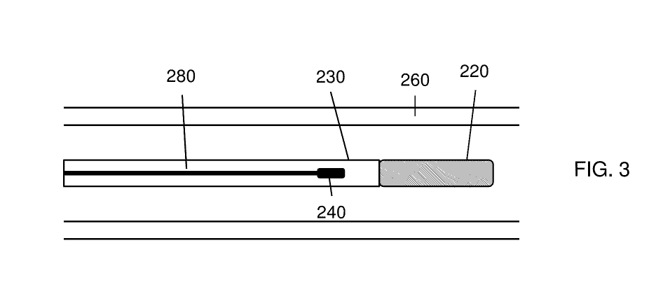

An embodiment of an imaging and treatment device within a lumen is shown in FIG. 3. The catheter body 230 is shown positioned within a cross section of a lumen 260 and includes an imaging assembly 240, a drive cable 280, and a denervation assembly 220. The drive cable 280 is disposed within the catheter body 230. The denervation assembly 220 is disposed on the catheter body 230. The imaging assembly 240 is disposed on the drive cable 280. It should be appreciated that the drive cable 280 and the imaging assembly 240 can be controlled by an actuator (not shown) to slide within the catheter body 230 and to rotate around the central axis of the drive cable or slide back and forth relative to the catheter body to enable the use of certain intraluminal imaging modalities described below such as pull-back IVUS or pull-back OCT.

In various embodiments, the imaging assembly may be integrated within the body of the catheter, circumscribe the catheter, be placed on a distal end face of the catheter, and/or run along the body of the catheter. The catheter may also include an outer support structure or coating surrounding the imaging assembly. Catheter bodies intended for intravascular introduction will typically have a length in the range from 50 cm to 200 cm and an outer diameter in the range from 1 French to 12 French (0.33 mm: 1 French), usually from 3 French to 9 French. In the case of coronary catheters, the length is typically in the range from 125 cm to 200 cm, the diameter is preferably below 8 French, more preferably below 7 French, and most preferably in the range from 2 French to 7 French.

### Imaging Assembly

In certain embodiments, the imaging and treatment device of the invention includes an imaging assembly. The imaging assembly may be disposed on the catheter body or on a drive cable depending on the imaging technology being employed. Any imaging assembly may be used with devices and methods of the invention, such as optical-acoustic imaging apparatus, intravascular ultrasound (IVUS) or optical coherence tomography (OCT). The imaging assembly is used to send and receive signals to and from the imaging surface that form the imaging data.

In some embodiments, the imaging assembly is an IVUS imaging assembly. The imaging assembly can be a phased-array IVUS imaging assembly, a pull-back type IVUS imaging assembly, including rotational IVUS imaging assemblies, or an IVUS imaging assembly that uses photoacoustic materials to produce diagnostic ultrasound and/or receive reflected ultrasound for diagnostics. IVUS imaging assemblies and processing of IVUS data are described for example in Yock, U.S. Pat. Nos. 4,794,931, 5,000,185, and 5,313,949; Sieben et al., U.S. Pat. Nos. 5,243,988, and 5,353,798; Crowley et al., U.S. Pat. No. 4,951,677; Pomeranz, U.S. Pat. No. 5,095,911, Griffith et al., U.S. Pat. No. 4,841,977, Maroney et al., U.S. Pat. No. 5,373,849, Born et al., U.S. Pat. No. 5,176,141, Lancee et al., U.S. Pat. No. 5,240,003, Lancee et al., U.S. Pat. No. 5,375,602, Gardineer et at., U.S. Pat. No. 5,373,845, Seward et al., Mayo Clinic Proceedings 71(7):629-635 (1996), Packer et al., Cardiostim Conference 833 (1994), “Ultrasound Cardioscopy,” Eur. J.C.P.E. 4(2):193 (June 1994), Eberle et al., U.S. Pat. No. 5,453,575, Eberle et al., U.S. Pat. No. 5,368,037, Eberle et at., U.S. Pat. No. 5,183,048, Eberle et al., U.S. Pat. No. 5,167,233, Eberle et at., U.S. Pat. No. 4,917,097, Eberle et at., U.S. Pat. No. 5,135,486, and other references well known in the art relating to intraluminal ultrasound devices and modalities. All of these references are incorporated by reference herein in their entirety.

IVUS imaging is widely used as a diagnostic tool for assessing a diseased vessel, such as an artery, within the human body to determine the need for treatment, to guide an intervention, and/or to assess its effectiveness. An IVUS device including one or more ultrasound transducers is introduced into the vessel and guided to the area to be imaged. The transducers emit and then receive backscattered ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a 360 degree cross-sectional image of the vessel where the device is placed.

There are two general types of IVUS devices in use today: rotational and solid-state (also known as synthetic aperture phased array). For a typical rotational IVUS device, a single ultrasound transducer assembly is located at the tip of a flexible driveshaft that spins inside a plastic sheath inserted into the vessel of interest. The transducer assembly is oriented such that the ultrasound beam propagates generally perpendicular to the axis of the device. The fluid-filled sheath protects the vessel tissue from the spinning transducer and driveshaft while permitting ultrasound signals to propagate from the transducer into the tissue and back. As the driveshaft rotates, the transducer is periodically excited with a high voltage pulse to emit a short burst of ultrasound. The same transducer then listens for the returning echoes reflected from various tissue structures. The IVUS imaging system assembles a two dimensional display of the vessel cross-section from a sequence of pulse/acquisition cycles occurring during a single revolution of the transducer. Suitable rotational IVUS catheters include, for example the REVOLUTION 45 MHz catheter (offered by the Volcano Corporation).

In contrast, solid-state IVUS devices carry a transducer complex that includes an array of ultrasound transducers distributed around the circumference of the device connected to a set of transducer controllers. The transducer controllers select transducer sets for transmitting an ultrasound pulse and for receiving the echo signal. By stepping through a sequence of transmit-receive sets, the solid-state IVUS system can synthesize the effect of a mechanically scanned transducer element but without moving parts. The same transducer elements can be used to acquire different types of intravascular data. The different types of intravascular data are acquired based on different manners of operation of the transducer elements. The solid-state scanner can be wired directly to the imaging system with a simple electrical cable and a standard detachable electrical connector.

The transducer subassembly can include either a single transducer or an array. The transducer elements can be used to acquire different types of intravascular data, such as flow data, motion data and structural image data. For example, the different types of intravascular data are acquired based on different manners of operation of the transducer elements. For example, in a gray-scale imaging mode, the transducer elements transmit in a certain sequence one gray-scale IVUS image. Methods for constructing IVUS images are well-known in the art, and are described, for example in Hancock et al. (U.S. Pat. No. 8,187,191), Nair et al. (U.S. Pat. No. 7,074,188), and Vince et al. (U.S. U.S. Pat. No. 6,200,268), the content of each of which is incorporated by reference herein in its entirety. In flow imaging mode, the transducer elements are operated in a different way to collect the information on the motion or flow. This process enables one image (or frame) of flow data to be acquired. The particular methods and processes for acquiring different types of intravascular data, including operation of the transducer elements in the different modes (e.g., gray-scale imaging mode, flow imaging mode, etc.) consistent with the present invention are further described in U.S. patent application Ser. No. 14/037,683, the content of which is incorporated by reference herein in its entirety.

The acquisition of each flow frame of data is interlaced with an IVUS gray scale frame of data. Operating an IVUS catheter to acquire flow data and constructing images of that data is further described in O'Donnell et al. (U.S. Pat. No. 5,921,931), U.S. Provisional Patent Application No. 61/587,834, and U.S. Provisional Patent Application No. 61/646,080, the content of each of which is incorporated by reference herein its entirety. Commercially available fluid flow display software for operating an IVUS catheter in flow mode and displaying flow data is CHROMAFLOW (IVUS fluid flow display software offered by the Volcano Corporation). Suitable phased array imaging assemblies are found on Volcano Corporation's EAGLE EYE Platinum Catheter, EAGLE EYE Platinum Short-Tip Catheter, and EAGLEEYE Gold Catheter.

The imaging guidewire of the present invention may also include advanced guidewire designs to include sensors that measure flow and pressure, among other things. For example, the FLOWIRE Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PrimeWire PRESTIGE pressure guidewire, available from Volcano Corp. (San Diego, Calif.), provides a microfabricated microelectromechanical (MEMS) pressure sensor for measuring pressure environments near the distal tip of the guidewire. Additional details of guidewires having MEMS sensors can be found in U.S. Patent Publication No. 2009/0088650, incorporated herein by reference in its entirety.

In addition to IVUS, other intraluminal imaging technologies may be suitable for use in methods of the invention for assessing and characterizing vascular access sites in order to diagnose a condition and determine appropriate treatment. For example, an Optical Coherence Tomography (OCT) catheter may be used to obtain intraluminal images in accordance with the invention. OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

OCT is a medical imaging methodology using a miniaturized near infrared light-emitting probe. As an optical signal acquisition and processing method, it captures micrometer-resolution, three-dimensional images from within optical scattering media (e.g., biological tissue). Recently it has also begun to be used in interventional cardiology to help diagnose coronary artery disease. OCT allows the application of interferometric technology to see from inside, for example, blood vessels, visualizing the endothelium (inner wall) of blood vessels in living individuals.

OCT systems and methods are generally described in Castella et al., U.S. Pat. No. 8,108,030, Milner et al., U.S. Patent Application Publication No. 2011/0152771, Condit et al., U.S. Patent Application Publication No. 2010/0220334, Castella et al., U.S. Patent Application Publication No. 2009/0043191, Milner et al., U.S. Patent Application Publication No. 2008/0291463, and Kemp, N., U.S. Patent Application Publication No. 2008/0180683, the content of each of which is incorporated by reference in its entirety.

In OCT, a light source delivers a beam of light to an imaging device to image target tissue. Light sources can include pulsating light sources or lasers, continuous wave light sources or lasers, tunable lasers, broadband light source, or multiple tunable laser. Within the light source is an optical amplifier and a tunable filter that allows a user to select a wavelength of light to be amplified. Wavelengths commonly used in medical applications include near-infrared light, for example between about 800 nm and about 1700 nm.

Aspects of the invention may obtain imaging data from an OCT system, including OCT systems that operate in either the time domain or frequency (high definition) domain. Basic differences between time-domain OCT and frequency-domain OCT is that in time-domain OCT, the scanning mechanism is a movable mirror, which is scanned as a function of time during the image acquisition. However, in the frequency-domain OCT, there are no moving parts and the image is scanned as a function of frequency or wavelength.

In time-domain OCT systems an interference spectrum is obtained by moving the scanning mechanism, such as a reference mirror, longitudinally to change the reference path and match multiple optical paths due to reflections within the sample. The signal giving the reflectivity is sampled over time, and light traveling at a specific distance creates interference in the detector. Moving the scanning mechanism laterally (or rotationally) across the sample produces two-dimensional and three-dimensional images.

In frequency domain OCT, a light source capable of emitting a range of optical frequencies excites an interferometer, the interferometer combines the light returned from a sample with a reference beam of light from the same source, and the intensity of the combined light is recorded as a function of optical frequency to form an interference spectrum. A Fourier transform of the interference spectrum provides the reflectance distribution along the depth within the sample.

Several methods of frequency domain OCT are described in the literature. In spectral-domain OCT (SD-OCT), also sometimes called “Spectral Radar” (Optics letters, Vol. 21, No. 14 (1996) 1087-1089), a grating or prism or other means is used to disperse the output of the interferometer into its optical frequency components. The intensities of these separated components are measured using an array of optical detectors, each detector receiving an optical frequency or a fractional range of optical frequencies. The set of measurements from these optical detectors forms an interference spectrum (Smith, L. M. and C. C. Dobson, Applied Optics 28: 3339-3342), wherein the distance to a scatterer is determined by the wavelength dependent fringe spacing within the power spectrum. SD-OCT has enabled the determination of distance and scattering intensity of multiple scatters lying along the illumination axis by analyzing a single the exposure of an array of optical detectors so that no scanning in depth is necessary. Typically the light source emits a broad range of optical frequencies simultaneously.

Alternatively, in swept-source OCT, the interference spectrum is recorded by using a source with adjustable optical frequency, with the optical frequency of the source swept through a range of optical frequencies, and recording the interfered light intensity as a function of time during the sweep. An example of swept-source OCT is described in U.S. Pat. No. 5,321,501.

Generally, time domain systems and frequency domain systems can further vary in type based upon the optical layout of the systems: common beam path systems and differential beam path systems. A common beam path system sends all produced light through a single optical fiber to generate a reference signal and a sample signal whereas a differential beam path system splits the produced light such that a portion of the light is directed to the sample and the other portion is directed to a reference surface. Common beam path systems are described in U.S. Pat. No. 7,999,938; U.S. Pat. No. 7,995,210; and U.S. Pat. No. 7,787,127 and differential beam path systems are described in U.S. Pat. No. 7,783,337; U.S. Pat. No. 6,134,003; and U.S. Pat. No. 6,421,164, the contents of each of which are incorporated by reference herein in its entirety.

In certain embodiments, angiogram image data is obtained simultaneously with the imaging data obtained from the imaging assembly and/or imaging guidewire of the present invention. In such embodiments, the catheter and/or guidewire may include one or more radiopaque labels that allow for co-locating image data with certain positions on a vasculature map generated by an angiogram. Co-locating intraluminal image data and angiogram image data is known in the art, and described in U.S. Publication Nos. 2012/0230565, 2011/0319752, and 2013/0030295.

In preferred embodiments, the imaging assembly is an optical-acoustic imaging apparatus. Optical-acoustic imaging apparatus include at least one imaging element to send and receive imaging signals. In one embodiment, the imaging assembly includes at least one acoustic-to-optical transducer. In certain embodiments, the acoustic-to-optical transducer is an Fiber Bragg Grating within an optical fiber. In addition, the imaging assemblies may include the optical fiber with one or more Fiber Bragg Gratings (acoustic-to-optical transducer) and one or more other transducers. The at least one other transducer may be used to generate the acoustic energy for imaging. Acoustic generating transducers can be electric-to-acoustic transducers or optical-to-acoustic transducers. The imaging assemblies suitable for use in devices of the invention are described in more detail below.

Fiber Bragg Gratings for imaging provides a means for measuring the interference between two paths taken by an optical beam. A partially-reflecting Fiber Bragg Grating is used to split the incident beam of light into two parts, in which one part of the beam travels along a path that is kept constant (constant path) and another part travels a path for detecting a change (change path). The paths are then combined to detect any interference in the beam. If the paths are identical, then the two paths combine to form the original beam. If the paths are different, then the two parts will add or subtract from each other and form an interference. The Fiber Bragg Grating elements are thus able to sense a change wavelength between the constant path and the change path based on received ultrasound or acoustic energy. The detected optical signal interferences can be used to generate an image using any conventional means.

In certain embodiments, the imaging assembly includes a piezoelectric element to generate the acoustic or ultrasound energy. In such aspect, the optical fiber of the imaging assembly may by coated by the piezoelectric element. The piezoelectric element may include any suitable piezoelectric or piezoceramic material. In one embodiment, the piezoelectric element is a poled polyvinylidene fluoride or polyvinylidene difluoride material. The piezoelectric element can be connected to one or more electrodes that are connected to a generator that transmits pulses of electricity to the electrodes. The electric pulses cause mechanical oscillations in the piezoelectric element, which generates an acoustic signal. Thus, the piezoelectric element is an electric-to-acoustic transducer. Primary and reflected pulses (i.e. reflected from the imaging medium) are received by the Bragg Grating element and transmitted to an electronic instrument to generate an imaging.

In some embodiments, the imaging assembly includes an optical fiber with Fiber Bragg Grating and a piezoelectric element. In this embodiment, an electrical generator stimulates the piezoelectric element (electrical-to-acoustic transducer) to transmit ultrasound impulses to both the Fiber Bragg Grating and the outer medium in which the device is located. For example, the outer medium may include blood when imaging a vessel. Primary and reflected impulses are received by the Fiber Bragg Grating (acting as an acoustic-to-optical transducer). The mechanical impulses deform the Bragg Grating and cause the Fiber Bragg Grating to modulate the light reflected within the optical fiber, which generates an interference signal. The interference signal is recorded by electronic detection instrument, using conventional methods. The electronic instrument may include a photodetector and an oscilloscope. An image can be generated from these recorded signals. The electronic instruments modulation of light reflected backwards from the optical fiber due to mechanical deformations. The optical fiber with a Bragg Grating described herein, the imaging assembly described herein and other varying embodiments are described in more detail in U.S. Pat. Nos. 6,659,957 and 7,527,594 and in U.S. Patent Publication No. 2008/0119739.

In another aspect, the imaging assembly does not require an electrical-to-acoustic transducer to generate acoustic/ultrasound signals. Instead, the imaging assembly utilizes the one or more Fiber Bragg Grating elements of the optical fiber in combination with an optical-to-acoustic transducer material to generate acoustic energy from optical energy. In this aspect, the acoustic-to-optical transducer (signal receiver) also acts as an optical-to-acoustic transducer (signal generator).

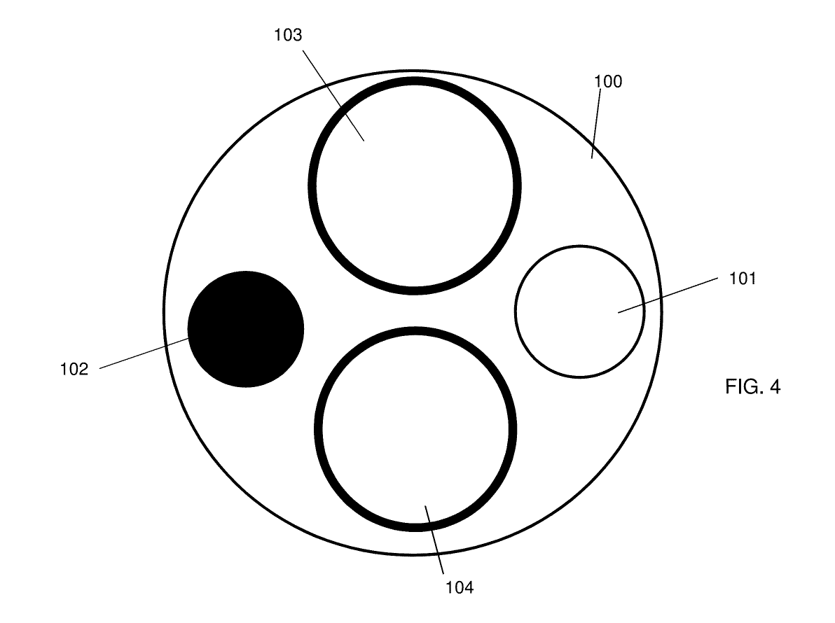

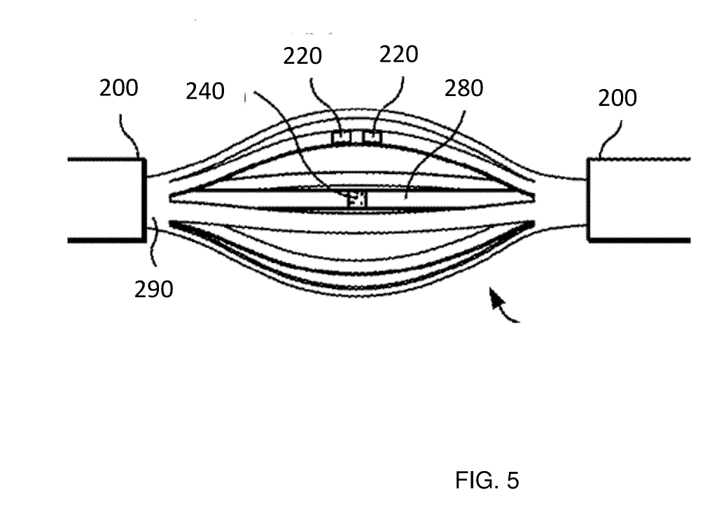

To generate the acoustic energy, imaging assembly may include a combination of blazed and unblazed Fiber Bragg Gratings. Unblazed Bragg Gratings typically include impressed index changes that are substantially perpendicular to the longitudinal axis of the fiber core of the optical fiber. Unblazed Bragg Gratings reflect optical energy of a specific wavelength along the longitudinal of the optical fiber. Blazed Bragg Gratings typically include obliquely impressed index changes that are at a non-perpendicular angle to the longitudinal axis of the optical fiber. Blazed Bragg Gratings reflect optical energy away from the longitudinal axis of the optical fiber. FIGS. 4 and 5 depict an imaging assembly according to this embodiment.

One or more imaging assemblies may be incorporated into an imaging guidewire or the catheter to allow an operator to image a luminal surface. The one or more imaging assemblies of the imaging guidewire or catheter are referred to generally as an imaging assembly. In some embodiments, instead of presenting one 2-D slice of the anatomy, the system is operated to provide a 3-D visual image that permits the viewing of a desired volume of the patient's anatomy or other imaging region of interest. This allows the physician to quickly see the detailed spatial arrangement of structures, such as lesions, with respect to other anatomy.

### Denervation Assembly

In a preferred embodiment, the imaging assembly of the invention may be combined with a denervation assembly on a single catheter. For example, a drive cable is introduced into the lumen of the catheter body and at least a portion of the drive cable is housed within the catheter body or lumen. The drive cable comprises an imaging assembly, capable of providing real-time imaging of the interior surface of the lumen wherein the catheter is disposed. A denervation assembly disposed on the catheter body may then release high intensity energy or apply cryotherapy via a refrigerant supplied to the distal portion of the catheter via an insulated intra-catheter lumen therein in order to effect denervation treatment to the target tissue.

In some embodiments, the denervation assembly comprises at least one transducer that generates high intensity ultrasound. High-Intensity Focused Ultrasound (HIFU, or sometimes FUS for Focused UltraSound) is a highly precise medical procedure that applies high-intensity focused ultrasound energy to locally heat and destroy diseased or damaged tissue through ablation. HIFU is a hyperthermia therapy, a class of clinical therapies that use temperature to treat diseases. HIFU is also one modality of therapeutic ultrasound, involving minimally invasive or non-invasive methods to direct acoustic energy into the body and at a tissue. In addition to HIFU, other modalities include ultrasound-assisted drug delivery, ultrasound hemostasis, ultrasound lithotripsy, and ultrasound-assisted thrombolysis. Clinical HIFU procedures are typically performed in conjunction with an imaging procedure to enable treatment planning and targeting before applying a therapeutic or ablative levels of ultrasound energy. When Magnetic resonance imaging (MRI) is used for guidance, the technique is sometimes called Magnetic Resonance-guided Focused Ultrasound, often shortened to MRgFUS or MRgHIFU. When diagnostic sonography is used, the technique is sometimes called Ultrasound-guided Focused Ultrasound (USgFUS or USgHIFU). Imaging and treatment devices of the invention allow for HIFU procedures without the need for externally applied imaging modalities during portions of the treatment procedure.

In one aspect, a denervation assembly is used to disrupt the nerves innervating a target tissue by delivering energy (RF energy, laser energy, etc.) within an artery to denude the nerve ends. In some embodiments, the denervation assembly includes at least one electrode. The electrodes can be arranged in many different patterns along the denervation assembly. In addition, the electrodes may have a variety of different shape and sizes. For example, the electrode can be a conductive plate, a conductive ring, conductive loop, or a conductive coil. In one embodiment, the at least one electrode includes a plurality of wire electrodes configured to extend out of the distal end of the imaging electrode.

The proximal end of the denervation assembly is connected to an energy source that provides energy to the electrodes for delivering high intensity energy. The energy necessary can be provided from a number of different sources including radiofrequency, laser, microwave, ultrasound and forms of direct current (high energy, low energy and fulgutronization procedures). Any source of energy is suitable for use in the denervation assembly of the invention. Preferably, the source of energy chosen does not disrupt the imaging of the vessel during the procedure with the imaging guidewire and/or imaging assembly.

In certain aspects, the denervation assembly comprises a thermally conductive member, in fluid communication with a refrigerant supply at the proximal portion of the catheter via an insulated lumen within the catheter body. The refrigerant supply lumen is insulated sufficiently to prevent the refrigerant from removing energy from the catheter body or the surrounding tissue. The thermally conductive denervation assembly permits the refrigerant to remove energy from the target tissue, in contact with the denervation assembly until the target nerve within the target tissue has been ablated. Refrigerant may then be removed from the catheter via a separate return lumen so that a fresh supply of refrigerant can be circulated to the denervation assembly. In various embodiments, the return lumen can be operably coupled to a pump (e.g., a vacuum pump, a DC-powered pump, etc.), a back-pressure control valve, and/or other suitable device. Any suitable refrigerant known in the art may be used. Suitable refrigerants include nitrous oxide (N2O), Argon (Ar), liquid nitrogen (N2), carbon dioxide (CO2), chlorofluorocarbon, hydrochlorofluorocarbon, or other hydrofluorocarbons. In other embodiments, for example, the supply of refrigerant can include a refrigerant known as R-410A, which is a near-azeotropic mixture of difluoromethane (CH2F2; known as R-32) and pentafluoroethane (CHF2CF3; known R-125). The refrigerant can be stored in a cartridge (e.g., a single-use cartridge) or a canister (e.g., a tank, cylinder, or other suitable containers that are not cartridges), which may be coupled to the proximal portion of the catheter. An exemplary embodiment of a catheter of the invention having a refrigerant based denervation assembly is shown in FIG. 4. FIG. 4 shows a cross section of a catheter 100 including a guidewire lumen 101, a drive cable 102, a refrigerant supply lumen 103, and a refrigerant return lumen 104 is shown in

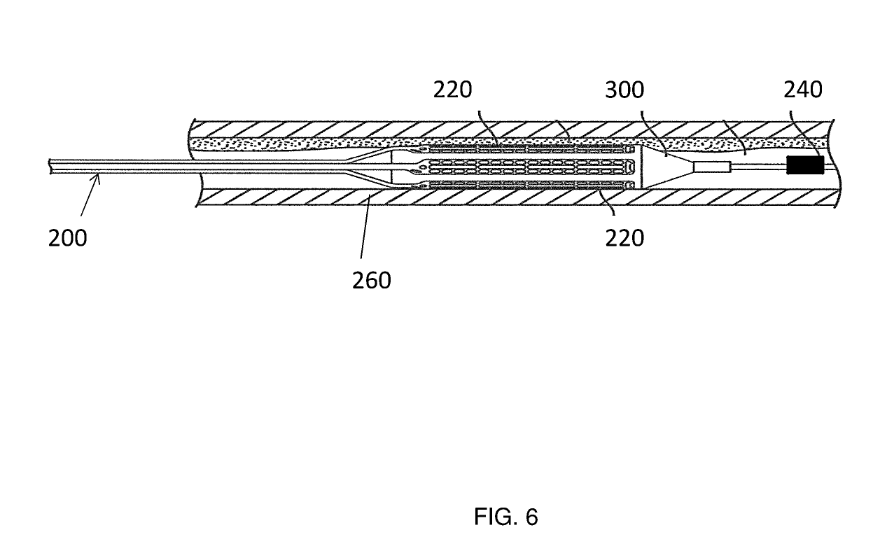

In certain embodiments, denervation assemblies of the invention must be brought into close proximity or direct contact with the target tissue. Accordingly, in various embodiments, catheters of the invention may have a denervation assembly disposed on an expandable member or disposed on a steerable tip of the catheter. The denervation assembly may comprise an expandable basket as shown in FIG. 5. FIG. 5 shows a catheter 200 with a drive cable 280 disposed within. One or more denervation assemblies 220 are disposed on the arms of an expandable basket 290 and an imaging assembly 240 is disposed on the drive cable. Alternatively, the catheter may comprise an expandable balloon as shown in FIG. 6. FIG. 6 shows a catheter 200 within a lumen 260 having denervation assemblies 220 disposed on an expandable balloon member 300. An imaging assembly 240 is disposed on the proximal end of the catheter body. The expandable balloon member 300 in FIG. 6 is shown in its expanded state so that the denervation assemblies 220 are in contact with the walls of the lumen 260.

In operation, the imaging portion of the device can be used to locate a treatment site within the vasculature that requires treatment. Once the treatment site is located, the denervation assembly is activated in the lumen of the catheter. The electrodes located on the distal end of the drive cable can be positioned and energized by an energy source operably associated with the electrodes. The energized electrodes deliver the energy to the tissue at the treatment site. In one embodiment, the imaging assembly of the catheter images the luminal surface and lumen during the treatment therapy. In an alternative embodiment, the denervation assembly deploys several rounds of treatment and the imaging assembly of the catheter is used to image the treated luminal surface between each round of energy.

In order to minimize risks when performing ablative procedures such as renal denervation (RDN), it is important to monitor and visualize the surrounding tissues. For example, during RDN, the renal artery could be weakened, increasing the chance of embolism, or the renal artery could be perforated or severed. To avoid such damage, prior art devices rely on gated energy delivery to control the temperature of the tissue. That is, RDN devices are programmed to provide predetermined dosing times and wattage based upon accumulated experience and animal/cadaver studies. For example, 4 Watts of radiofrequency energy delivered for 2 seconds has been found to increase the temperature of a cadaver aorta to 65° C. with a particular balloon ablation device. See U.S. Patent Publication No. 2012/0158101 incorporated by reference herein in its entirety. Operation within the suggested range is assumed to provide safe and effective treatment. Nonetheless, without active monitoring of the treatment site, it is impossible to know if the renal artery tissue is being over treated. Using prior art methods, it is impossible to determine if the tissue has been adequately denervated without prolonged blood pressure monitoring after the procedure.

In some aspects, the transducers may comprise capacitive micromachined ultrasonic transducers (CMUTs). CMUTs, which uses micromachining technology, allows for miniaturize device dimensions and produces capacitive transducers that perform comparably to the piezoelectric counterparts. CMUTs are essentially capacitors with one moveable electrode. If an alternating voltage is applied to the device then the moveable electrode begins to vibrate, thus causing ultrasound to be generated. If the cMUTs are used as receivers, then changes in pressure such as those from an ultrasonic wave cause the moveable electrode to deflect and hence produce a measurable change in capacitance. See for example, Ergun et al., Journal of Aerospace Eng., April 2003, 16:2(76) page 76-84. CMUT arrays can be made in any arbitrary geometry with very small dimensions using photolithographic techniques and standard microfabrication processes. See Khuri-Yakub et al. J Micromech Microeng. May 2011; 21(5): 054004-054014.

In some aspects, the transducers may comprise piezoelectric micromachined ultrasonic transducers (pMUTs), which are based on the flexural motion of a thin membrane coupled with a thin piezoelectric film. See for example Trolier-McKinstry, Susan; P. Muralt (January 2004). “Thin Film Piezoelectric for MEMS”. Journal of Electroceramics 12 (1-2): 7. doi:10.1023/B:JECR.0000033998.72845.51. It should be noted that pMUTs exhibit superior bandwidth and offer considerable design flexibility, which allows for operation frequency and acoustic impedance to be tailored for numerous applications.

In a preferred embodiment, the device of the invention is positioned in the renal artery of a patient. Using a computer system, the array of transducers located on the catheter body image the interior lumen of the renal artery to thereby display in real time at least a portion of the renal artery on a monitor. The user is able to locate a region of interest and once selected, activate the denervation assembly on the distal portion of the catheter to deliver high energy to the region of interest. The user is then able to further view the region of interest to determine whether subsequent applications of energy is needed or required.

Other embodiments of catheters and systems of using them, not disclosed herein, will be evident to those of skill in the art, and are intended to be covered by the claims listed below.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An imaging and treatment device comprising:
a catheter body having a distal portion, a proximal portion, and a lumen disposed within;
an intravascular imaging assembly on the distal portion of the catheter body; and
a denervation assembly adjacent to the imaging assembly on the distal portion of the catheter body and configured to remove energy from a target tissue via a refrigerant supplied to the denervation assembly through the catheter lumen.

2. The imaging and treatment device of claim 1, wherein the catheter body comprises an additional lumen for receiving a guidewire.

3. The imaging and treatment device of claim 1, wherein the imaging assembly comprises an intravenous ultrasound (IVUS) imaging assembly or an optical coherence tomography (OCT) imaging assembly.

4. The imaging and treatment device of claim 3, wherein the IVUS imaging assembly comprises piezoelectric micromachined ultrasonic transducers (PMUT) or capacitive micromachined ultrasonic transducers (CMUT).

5. The imaging and treatment device of claim 1, wherein the catheter lumen supplying refrigerant is insulated so that the refrigerant does not remove energy from the catheter body.

6. The imaging and treatment device of claim 1, wherein the denervation assembly comprises a thermally conductive surface, in fluid communication with the catheter lumen supplying refrigerant so that the refrigerant is able to remove energy from the target tissue where the denervation assembly contacts said target tissue.

7. The imaging and treatment device of claim 1, wherein the denervation assembly is disposed on an expandable member so that, when expanded, the denervation assembly may be brought into direct contact with the target tissue.

8. The imaging and treatment device of claim 1, wherein the device further comprises a controller configured to:
cause the intravascular imaging assembly on the catheter body to generate a plurality of data;
receive the data;
display an image that comprises the data; and
circulate the refrigerant to the denervation assembly.

9. An imaging and treatment device comprising:
a catheter body having a distal portion, proximal portion, and a lumen disposed within;
a drive cable having an intravenous ultrasound (IVUS) imaging assembly disposed at a distal region of the drive cable and configured to be placed slidably within the lumen of the catheter; and
a denervation assembly disposed at the distal portion of the catheter body.

10. The imaging and treatment device of claim 9, wherein the catheter body comprises an additional lumen for receiving a guidewire.

11. The imaging and treatment device of claim 9, wherein the catheter body comprises a steerable distal tip with the denervation assembly disposed thereon so that the denervation assembly may be brought into direct contact with the target tissue via a control mechanism attached to the proximal portion of the catheter body.

12. The imaging and treatment device of claim 9, wherein the denervation assembly comprises an expandable member so that, when expanded, the denervation assembly may be brought into direct contact with the target tissue.

13. The imaging and treatment device of claim 9, wherein the denervation assembly comprises one or more electrodes configured to apply radiofrequency energy to the target tissue.

14. The imaging and treatment device of claim 9, wherein the denervation assembly comprises one or more transducers configured to apply high intensity focused ultrasound energy to the target tissue.

15. The imaging and treatment device of claim 9, wherein the denervation assembly is configured to remove energy from a target tissue via a refrigerant supplied to the denervation assembly through an additional lumen in the catheter.

16. The imaging and treatment device of claim 9, wherein the IVUS imaging assembly comprises piezoelectric micromachined ultrasonic transducers (PMUT) or capacitive micromachined ultrasonic transducers (CMUT).

17. The imaging and treatment device of claim 9, wherein the device further comprises a comprises a controller configured to:
manipulate the drive cable so that the IVUS assembly is rotated relative to the catheter body and slid back within the lumen of the catheter away from the distal portion of the catheter body;
cause the IVUS assembly on the drive cable to generate a plurality of data while being rotated and slid back;
receive the data;
display an image that comprises the data; and
activate the denervation assembly.

18. An imaging and treatment device comprising:
a catheter body having a distal portion and a proximal portion;
a drive cable having an optical coherence tomography (OCT) imaging assembly disposed at a distal region of the drive cable and configured to be placed slidably within the lumen of the catheter; and
a denervation assembly disposed at the distal portion of the catheter body.

19. The imaging and treatment device of claim 18, wherein the catheter body comprises an additional lumen for receiving a guidewire.

20. The imaging and treatment device of claim 18, wherein the catheter body comprises a steerable distal tip with the denervation assembly disposed thereon so that the denervation assembly may be brought into direct contact with the target tissue via a control mechanism attached to the proximal portion of the catheter body.

21. The imaging and treatment device of claim 18, wherein the denervation assembly comprises an expandable member so that, when expanded, the denervation assembly may be brought into direct contact with the target tissue.

22. The imaging and treatment device of claim 18, wherein the denervation assembly comprises one or more electrodes configured to apply radiofrequency energy to the target tissue.

23. The imaging and treatment device of claim 18, wherein the denervation assembly comprises one or more transducers configured to apply high intensity focused ultrasound energy to the target tissue.

24. The imaging and treatment device of claim 18, wherein the denervation assembly is configured to remove energy from a target tissue via a refrigerant supplied to the denervation assembly through an additional lumen in the catheter.

25. The imaging and treatment device of claim 18, wherein the device further comprises a comprises a controller configured to:
manipulate the drive cable so that the OCT assembly is rotated relative to the catheter body and slid back within the lumen of the catheter away from the distal portion of the catheter body;
cause the OCT assembly on the drive cable to generate a plurality of data while being rotated and slid back;
receive the data;
display an image that comprises the data; and
activate the denervation assembly.
